(19) United States
(12) Patent Application Publication
(10) Pub. No.: US 2022/0233631 A1
(43) Pub. Date: Jul. 28, 2022

(54) STABLE FORMULATION OF CETRORELIX

(71) Applicant: INTAS PHARMACEUTICALS LTD., Ahmedabad, Gujarat (IN)

(72) Inventors: Alex K GEORGE, Sanand Ahmedabad, Gujarat (IN); Shailesh Kumar JAIN, Sanand Ahmedabad, Gujarat (IN)

(73) Assignee: INTAS PHARMACEUTICALS LTD., Ahmedabad, Gujarat (IN)

(21) Appl. No.: 17/619,502

(22) PCT Filed: Jun. 16, 2020
(86) PCT No.: PCT/IB2020/055604

§ 371 Date: Dec. 15, 2021

(30) Foreign Application Priority Data
Jun. 17, 2019 (IN) 201921023926

(51) Int. Cl. A61K 38/09; A61K 31/222; A61K 47/12; A61K 47/26; A61K 47/69; A61K 9/00

(52) U.S. Cl. CPC A61K 38/09; A61K 31/222; A61K 9/0019; A61K 47/26; A61K 47/6951; A61K 47/12

(57) ABSTRACT

The present invention relates to a stable formulation of Cetrorelix or its pharmaceutically acceptable salt in the form of ready-to-use solution. The said stable ready-to-use solution of Cetrorelix prevents gel formation and provides better patient compliance. Further, the invention relates to a process for preparation of the said stable ready-to-use solution of Cetrorelix.

### RELATED APPLICATIONS

This application is related to Indian Provisional Application No. IN201921023926 filed on 17 Jun. 2019 and is incorporated herein in its entirety.

### FIELD OF THE INVENTION

The present invention relates to a stable formulation of Cetrorelix or its pharmaceutically acceptable salt. The said stable formulation is in the form of ready-to-use solution. Also, the said stable ready-to-use solution of Cetrorelix prevents formulation related issues like gel formation; and provides better patient compliance. Further, the invention relates to a process for preparation of the said stable ready-to-use solution of Cetrorelix. Moreover, the invention relates to a pre-filled ready-to-use device comprising said stable ready-to-use solution of Cetrorelix.

### BACKGROUND OF THE INVENTION

Chemically, Cetrorelix is gonadotropin releasing hormone (GnRH) antagonist acetyl-D-3-(2′-naphtyl-alanine-D-4-chlorophenylalanine-D-3-(3′-pyridyl)-alanine- L-serine-L-tyrosine-D-citruline-L-leucine-L-arginine-L-proline-D-alanine-amide (C₇₀H₉₂C₁N₁₇O₁₄) having the following formula:

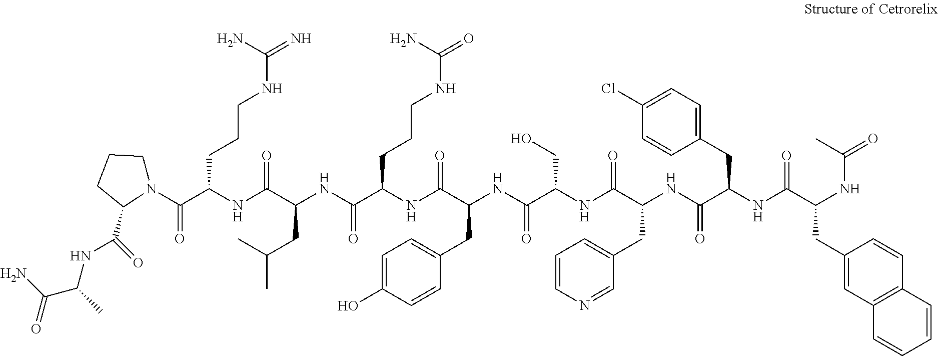 [SMILES: CC(=O)N[C@H](Cc1ccc2ccccc2c1)C(=O)N[C@H](Cc1ccc(Cl)cc1)C(=O)N[C@H](Cc1cccnc1)C(=O)N[C@@H](CO)C(=O)N[C@@H](Cc1ccc(O)cc1)C(=O)N[C@H](CCCNC(N)=O)C(=O)N[C@@H](CC(C)C)C(=O)N[C@@H](CCCNC(=N)N)C(=O)N1CCC[C@H]1C(=O)N[C@H](C)C(N)=O]

Cetrorelix is a decapeptide with a terminal acid amide group. Cetrorelix was earlier disclosed in US4800191 patent. Cetrorelix acetate is currently been marketed as a

Cetrotide® lyophilized powder by EMD Serono Inc. The sterile lyophilized powder is intended for subcutaneous injection after reconstitution with sterile water for injection. Cetrotide® is used for the inhibition of premature luteinizing hormone (LH) surges in women undergoing controlled ovarian stimulation.

The currently marketed Cetrotide® is present only in the form of lyophilized powder in glass vials and reconstitution is necessary before subcutaneous administration. The said reconstitution process is accomplished by injecting a diluent into said glass vials which contain Cetrorelix. According to Cetrotide® summary of product characteristics (SmPC), the obtained reconstituted solution should be reconstituted using a gentle, swirling motion, and vigorous shaking with bubble formation should be avoided. Consequently, the said reconstitution process is a high time-consuming event, and hence it might hinder patient treatment compliance.

The formulation aspects of developing a stable formulation of Cetrorelix acetate are hindered by the fact that oligopeptides particularly having a terminal acid amide group are prone to gel formation.

The PCT patent application W02012077131 discloses a stable ready-to-use aqueous pharmaceutical preparation containing Cetrorelix or its pharmaceutically acceptable salt, wherein the preparation does not contain any surfactant and cyclodextrin. The concentration of glacial acetic acid was 4.5 mg/ml, and the pH of the said preparation was obtained in the preferred range of 2.8 to 3.5. However, this Cetrorelix preparation was found to result into severe pain at the injection site, and therefore it was not acceptable product with regards to the patient compliance.

The patent CA2115943 discloses a process for the preparation of Cetrorelix lyophilisate. It also discloses that aqueous solutions of the decapeptide cannot be autoclaved because these aqueous solutions of the decapeptide are unstable. During sterile filtration of the bulk solution, formation of gels on the sterile filter would increase the viscosity of the solution and thereby hinder the filtration step.

However, the bulk solution cannot be administered as a ready-to-use injection solution as such because of the following limitations:

hypertonic nature of the solution which may damage the local site e.g., irritation, edema, swelling, redness, etc.

high quantity of acetic acid is above safety level for subcutaneous/parenteral route of administration.

The patent US7718599 relates to pharmaceutical compositions suitable for parenteral administration comprising peptides in the form of acetate, gluconate, glucuronate, lactate, citrate, ascorbate, benzoate or phosphate salts in dissolved or dispersed form and at least one of the acid selected from a group of gluconic acid, glucaric acid or galactouronic acid for forming the salts in free acid form. The pharmaceutically acceptable acids are capable of imparting a pH in between 2.5 to 4.5 to the composition which helps in suppressing aggregation of Cetrorelix acetate. Further, the patent discloses lyophilized aggregation-free Cetrorelix preparation containing various cyclodextrins.

The patent US7214662 discloses aqueous injectable solution of Cetrorelix in an organic acid, such as gluconic acid. Further, the patent also mentioned that use of surfactant reduces the tendency of LHRH substances to aggregate. The patent application US20170189536 discloses the use of cyclodextrin and surfactant in aqueous formulation of polypeptide such as antibodies.

Although there are references with regards to ready-to-use solution comprising Cetrorelix, the formulation of stable ready-to-use solution is very challenging.

Therefore, there exists a need for providing a stable formulation of Cetrorelix which can be administered as a ready-to-use solution, wherein the pharmaceutical preparation can be conveniently prepared and sterilized by filtration.

The inventors of the present invention have developed a stable aqueous ready-to-use solution of Cetrorelix, which would overcome the formulation difficulties such as gel formation and also provide better patient compliance.

The present invention provides a stable ready-to-use solution comprising Cetrorelix or its pharmaceutically acceptable salt, a cyclodextrin, glacial acetic acid and a surfactant.

### OBJECTS OF THE INVENTION

The main object of the invention is to provide a stable formulation comprising Cetrorelix or its pharmaceutically acceptable salt, a cyclodextrin, glacial acetic acid and a surfactant.

Another object of the invention is to provide a stable formulation comprising Cetrorelix or its pharmaceutically acceptable salt, a cyclodextrin, glacial acetic acid and a surfactant; wherein the said formulation can be administered as a ready-to-use solution.

Another object of the invention is to provide a stable formulation comprising Cetrorelix or its pharmaceutically acceptable salt, a cyclodextrin, glacial acetic acid and a surfactant, wherein the pH of the said formulation is from about 3.5 to 8.5.

Another object of the invention is to provide a stable formulation comprising Cetrorelix or its pharmaceutically acceptable salt, a cyclodextrin, glacial acetic acid and a surfactant, wherein the said formulation remains as a clear solution without gel formation.

Another object of the invention is to provide a stable formulation comprising Cetrorelix or its pharmaceutically acceptable salt, a cyclodextrin, glacial acetic acid and a surfactant; wherein the assay of Cetrorelix after filter sterilization of the said formulation is not less than 95% of the assay of Cetrorelix before filter sterilization.

Another object of the present invention is to provide a process for preparation of a stable formulation comprising Cetrorelix or its pharmaceutically acceptable salt, a cyclodextrin, glacial acetic acid and a surfactant.

### SUMMARY OF THE INVENTION

In a first embodiment, the present invention relates to a stable formulation comprising Cetrorelix or its pharmaceutically acceptable salt, a cyclodextrin, glacial acetic acid and a surfactant.

In another embodiment, the present invention relates to a stable formulation comprising Cetrorelix or its pharmaceutically acceptable salt, a cyclodextrin, glacial acetic acid and a surfactant; wherein the said formulation can be administered as a ready-to-use solution.

In another embodiment, the present invention relates to a stable formulation comprising Cetrorelix or its pharmaceutically acceptable salt, a cyclodextrin, glacial acetic acid and a surfactant, wherein the pH of the said formulation is from about 3.5 to 8.5.

In another embodiment, the present invention relates to a stable formulation comprising Cetrorelix or its pharmaceutically acceptable salt, Hydroxypropyl beta Cyclodextrin (HP-β-CD), Polysorbate 80, glacial acetic acid, mannitol, and water for injection, wherein the said formulation remains as a clear solution without gel formation.

In another embodiment, the present invention relates to a stable formulation comprising Cetrorelix or its pharmaceutically acceptable salt, a cyclodextrin, glacial acetic acid and a surfactant; wherein the assay of Cetrorelix after filter sterilization of the said formulation is not less than 95% of the assay of Cetrorelix before filter sterilization.

In another embodiment, the present invention relates to a process for preparation of a stable formulation; wherein the process comprises the steps of:
a) Transferring the water for injection into stainless steel vessel, and sparging nitrogen gas into the water.
b) Adding Hydroxypropyl beta Cyclodextrin to the solution and stiffing the solution,
c) Adding Mannitol, Polysorbate 80, and Cetrorelix acetate to the above bulk solution with stiffing until it gets dissolved,
d) Adjusting the desired pH of the bulk solution with glacial acetic acid,
e) Filtering the bulk solution, and filling the sterile solution into pre-filled syringe or vial or pen or another device.

### DETAILED DESCRIPTION

The detailed description and the examples provided herein are exemplary and any modification or variation within the scope of the invention will be apparent to a person skilled in the art. Further, unless otherwise defined, all the technical and scientific terms used herein shall bear the meaning as understood by a person who is ordinarily skilled in the art.

In one embodiment, the present invention provides a stable formulation comprising Cetrorelix or its pharmaceutically acceptable salt, cyclodextrin, glacial acetic acid and a surfactant. The stable formulation of Cetrorelix can be administered as a ready-to-use solution. The administration of Cetrorelix solution can be done as subcutaneous, intravenous, or intramuscular injection. In a preferred embodiment, the Cetrorelix solution can be administered as a subcutaneous injection.

The sterilization of the peptides is challenging, and the heat sterilization can degrade the proteins and cause loss of potency. Hence, the filter sterilization can be used for the preparation of the peptide injections. However, during sterile filtration of the bulk solution, formation of gels on the filter would increase the viscosity of the solution and hence hinder the filtration step. Therefore, the inventors of the present invention have developed a stable formulation of Cetrorelix such that it remains aggregation-free clear colourless solution that can be readily filter-sterilized and there is no loss of potency after filter sterilization.

The term “stable formulation” of the present invention means the Cetrorelix solution that remains aggregation-free, clear colourless solution and the assay of Cetrorelix after filter sterilization (filtered bulk) of the said formulation is not less than 95% of the assay of Cetrorelix before filter sterilization (unfiltered bulk). The formulation can remain stable under the storage conditions of 2° -8° C. and 25° C./60% RH (relative humidity) for at least 1 month, preferably for at least 6 months, and more preferably for at least 12 months.

The inventors of the present invention studied the impurity profile of the lyophilized product (Cetrotide®) as well as the ready-to-use (RTU) Cetrorelix formulation, since both the lyophilized and ready-to-use products were intended to be stored at 2° -8° C.

It is known from the literature that peptides may undergo hydrolysis. Cetrorelix undergoes hydrolysis at the terminal amide group to produce impurity-C of Cetrorelix. Hence, for Cetrorelix acetate injection, in particular, the control of the impurity-C was required. In one embodiment, the impurity-C of Cetrorelix is below 0.50% under the storage conditions of 2° -8° C. and 25° C./60% RH for at least six months.

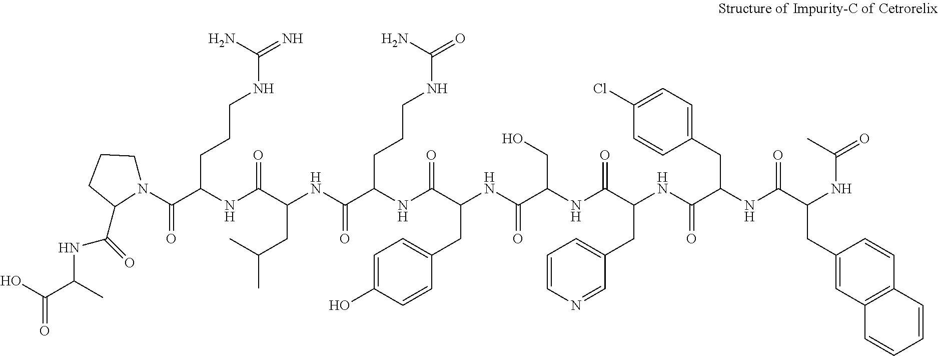 [SMILES: CC(=O)NC(Cc1ccc2ccccc2c1)C(=O)NC(Cc1ccc(Cl)cc1)C(=O)NC(Cc1cccnc1)C(=O)NC(CO)C(=O)NC(Cc1ccc(O)cc1)C(=O)NC(CCCNC(N)=O)C(=O)NC(CC(C)C)C(=O)NC(CCCNC(=N)N)C(=O)N1CCCC1C(=O)NC(C)C(=O)O]

The impurity-C refers to (R)-2-((S)-1-((2S, 5S, 8R, 11S, 14S, 17R, 20R, 23R)-20-(4- Chlorobenzyl)-2-(3-guanidinopropyl)-11-(4-hydroxybenzyl)-14-(hydroxymethyl)-5- isobutyl-23-(naphthalen-2-ylmethyl)-4,7,10,13,16,19,22,25-octaoxo-17-(pyridin-3- ylmethyl)-8-(3-ureidopropyl)-3,6,9,12,15,18,21,24-octaazahexacosan-1-oyl)pyro- lidine-2-carboxamido)propanoic acid.

It was found that the stable ready-to-use (RTU) Cetrorelix formulation of the present invention exhibited better impurity profile as compared to the reference lyophilized product (Cetrotide®). In particular, the impurity-C was controlled below quantification level in Cetrorelix RTU formulation as compared to lyophilized reference product. It was observed that the impurity-C level was increased in lyophilized product kept under the storage conditions of 2° -8° C. and 25° C./60% RH for at least six months; however, the impurity-C level remained below quantification level under the same storage conditions for the Cetrorelix formulation of the present invention. Hence, the impurity-C was better controlled and stabilized in Cetrorelix RTU formulation of the present invention as compared to the lyophilized product. In one embodiment, the impurity-C of Cetrorelix is below 0.50% for at least 6 months under the storage conditions of 2° -8° C.

In a preferred embodiment, the active ingredient is Cetrorelix acetate. The concentration of Cetrorelix or its pharmaceutically acceptable salt is in the concentration of about 0.01% to 10% W/V. In a preferred embodiment, the concentration of Cetrorelix acetate is about 0.05% WN. Further, the amount of the active ingredient in the formulation can be varied that is within the scope of a person skilled in the art. In a preferred embodiment the volume of said aqueous pharmaceutical composition is between 0.25 to 1 ml, preferably about 0.5 ml.

In another embodiment, the present invention provides a stable formulation comprising Cetrorelix or its pharmaceutically acceptable salt, wherein the pH of the said formulation is from about 3.5 to 8.5, preferably from about 4 to 6, and more preferably at about pH 5.

The inventors of the present invention observed that the prior art formulations of cetrorelix disclosed in the PCT patent application W02012077131 caused severe pain at the injection site. One of the reasons for the lack of patient compliance of the prior art formulation is the higher amount of glacial acetic acid (i.e. 4.5 mg/ml) and lower pH (i.e. below 3.5). The cetrorelix formulations of the present invention have pH from about 3.5 to 8.5 and lower amount of glacial acetic acid (i.e. not more than 0.05 μg/ml). Further, the cetrorelix formulations of the present invention comprises cyclodextrin and surfactant, which proved advantageous to provide excellent stability to the said formulations.

The stable formulation of the present invention can comprise different types of cyclodextrin selected from the group of α-Cyclodextrin, β-Cyclodextrin, γ-Cyclodextrin, HP-β-CD (Hydroxypropyl beta Cyclodextrin), Sulfobutyl- Ether-βCyclodextrin (SBE-β-CD) or the likes thereof. The preferred cyclodextrin used in the stable formulation of Cetrorelix or its pharmaceutically acceptable salt is HP-β-CD

(Hydroxypropyl beta Cyclodextrin). The amount of cyclodextrin in the formulation can be from about 0.1% to 30% W/V, preferably from about 5% to 25% W/V, and more preferably as 20% W/V HP-β-CD.

The stable formulation of the present invention further comprises a surfactant. The surfactant prevents aggregation by decreasing the surface tension. The surfactant is selected from Polysorbate 80, Polysorbate 60, Polysorbate 20 or the likes thereof. The preferred surfactant used in the stable formulation of Cetrorelix or its pharmaceutically acceptable salt is Polysorbate 80. The amount of Polysorbate in the formulation can be from about 0.001% to 1% W/V, preferably from about 0.001% to 0.05%, and more preferably from 0.001 to 0.01% W/V.

The stable formulation of the present invention can additionally comprise tonicity adjusting agent, which can decrease the hemolysis of blood cells and reduce pain at the injection site. The tonicity adjusting agent is selected from Mannitol, Dextrose, Lactose, Sucrose, or the likes thereof. The preferred tonicity adjusting agent for the present invention is mannitol. The amount of tonicity adjusting agent in the formulation can be from 0 to 10% W/V, preferably from 0.5 to 5% W/V, and more preferably from 1 to 3% W/V mannitol.

Further, other pharmaceutical excipients that can be optionally used in the present invention include chelating agents, buffers, pH adjusters, antioxidants and preservatives.

In another embodiment, the present invention relates to a stable formulation of cetrorelix comprising cetrorelix acetate, hydroxypropyl beta cyclodextrin, polysorbate 80, glacial acetic acid, mannitol, and water for injection.

In a preferred embodiment, the present invention relates to a stable formulation comprising 0.5% W/V cetrorelix acetate, 20% W/V hydroxypropyl beta cyclodextrin (HP-β-CD), 0.001% W/V polysorbate 80, 2% W/V mannitol, glacial acetic acid (to adjust the pH), and water for injection, wherein the pH of the said formulation is from about 3.5 to 8.5.

The ready-to-use formulation of the invention can be supplied in into a suitable container e.g., ampoule, vial, or a prefilled device such as prefilled syringe or pen, preferably the prefilled device is a glass prefilled syringe.

In another embodiment, the present invention provides a process for the preparation of a stable formulation; wherein the process comprises the steps of:
a) Transferring the water for injection into stainless steel vessel, and sparging nitrogen gas into the water.
b) Adding Hydroxypropyl beta Cyclodextrin to the solution and stiffing the solution,
c) Adding Mannitol, Polysorbate 80, and Cetrorelix acetate to the above bulk solution with stiffing until it gets dissolved,
d) Adjusting the desired pH of the bulk solution with glacial acetic acid,
e) Filtering the bulk solution, and filling the sterile solution into a suitable container e.g., ampoule, vial, pre-filled syringe, pen or another device.

Further, an inert gas sparging (nitrogen gas) can be carried out in any of the steps of the preparation process. The modifications in the preparation process can be made as known to the person skilled in the art.

The assay of Cetrorelix according to the present invention can be carried out by any of the methods known to a person skilled in the art, e.g. High performance liquid chromatography (HPLC method), Spectrophotometry (UV spectrophotometry), etc. The HPLC method was applied for performing the assay studies.

### Examples

The present invention has been described by way of example only, and it is to be recognized that modifications thereto falling within the scope and spirit of the appended claims, and which would be obvious to a person skilled in the art based upon the disclosure herein, are also considered to be included within the scope of this invention.

### Example-1

### Stable formulation of Cetrorelix.

| No. | Ingredients | Quantity (% w/v) |
|---|---|---|
| 1 | Cetrorelix acetate equivalent to Cetrorelix | 0.01-10% |
| 2 | Cyclodextrin | 0.1-30% |
| 3 | Tonicity agent | 0-10% |
| 4 | Surfactant | 0.001-1.0% |
| 5 | Glacial acetic acid* | q.s. to pH adj. |
| 6 | Water for Injection | q.s. to 100% |

*Not more than 0.05 μg/ml (i.e. 0.025 μg/0.5 ml).

Manufacturing process:

a) Transferring the water for injection into stainless steel vessel, and sparging nitrogen gas into the water,

b) Adding Cyclodextrin to the solution and stirring the solution,

c) Adding tonicity agent, surfactant, and Cetrorelix acetate to the above obtained solution with stirring until it gets dissolved,

d) Adjusting the desired pH of the solution with glacial acetic acid,

e) Filtering the solution, and filling the sterile solution into prefilled syringes.

### Example-2

### Stable formulation of Cetrorelix (0.5 mg/ml).

| | | Quantity | | Quantity |
|---|---|---|---|---|
| No. | Ingredients | mg/ml | | (% w/v) |
| 1 | Cetrorelix acetate equivalent to | 0.5 | mg | 0.05% |
| | Cetrorelix | | | |
| 2 | Hydroxypropyl Beta | 200 | mg | 20% |
| | cyclodextrin | | | |
| 3 | Mannitol | 20 | mg | 2% |
| 4 | Polysorbate 80 | 0.01 | mg | 0.001% |

| | | | |
|---|---|---|---|
| 5 | Glacial acetic acid* | q.s. to pH adj. | q.s. to pH adj. |
| 6 | Water for Injection | q.s. to 1.0 ml | q.s. to 100% |

*Not more than 0.05 μg/ml (i.e. 0.025 μg/0.5 ml).

The Example-2 can be prepared by the process as described in the above Example-1.

The assay result of the unfiltered and filtered bulk is given in the below table:

| No. | Test | Unfiltered Bulk | Filtered Bulk |
|---|---|---|---|
| 1 | Description | Clear colourless solution | Clear colourless solution |
| 2 | Assay | 102.3% | 102.7% |

The visual observations and the assay results indicate that the formulation of Cetrorelix acetate remains aggregation-free, clear colourless solution and the assay of Cetrorelix after filter sterilization is not less than 95%.

### EXAMPLE-3

### Stable formulation of Cetrorelix (0.25 mg/0.5 ml).

| | | Quantity | | Quantity |
|---|---|---|---|---|
| No. | Ingredients | mg/0.5 ml | | (% w/v) |
| 1 | Cetrorelix acetate equivalent to | 0.25 | mg | 0.05% |
| | Cetrorelix | | | |
| 2 | Hydroxypropyl Beta | 100 | mg | 20% |
| | cyclodextrin | | | |
| 3 | Mannitol | 10 | mg | 2% |
| 4 | Polysorbate 80 | 0.005 | mg | 0.001% |

| | | | |
|---|---|---|---|
| 5 | Glacial acetic acid* | q.s. to pH 5.0 | q.s. to pH 5.0 |
| 6 | Water for Injection | q.s. to 0.5 ml | q.s. to 100% |

*Not more than 0.05 μg/ml (i.e. 0.025 μg/0.5 ml).

The Example-3 (Cetrorelix acetate Injection; 0.25 mg/0.5 ml) can be prepared by the process as described in the Example-1.

The stability data of Cetrorelix acetate Injection 0.25 mg/0.5 ml upon storage at 25° C./60% RH conditions for at least one month is as follows:

| No. | Test | Unfiltered Bulk | Filtered Bulk |
|---|---|---|---|
| 1 | Description | Clear colourless solution | Clear colourless solution |
| 2 | Assay | 102.7% | 103.2% |
| 3 | pH | 4.98 | 4.92 |

The visual observations and the assay results indicate that the formulation of

Cetrorelix acetate remains aggregation-free, clear colourless solution and the assay of Cetrorelix after filter sterilization is not less than 95%. Therefore, the stable formulation of Cetrorelix can be used as a ready-to-use solution and provides better patient compliance.

### EXAMPLE-4:

### Stability results for Cetrorelix formulation.

The stability data of cetrorelix acetate Injection of the Example-3 upon storage at 2° -8° C. and 25 ° C./60% RH conditions for at least six months is as follows:

| | | 25° C./60% RH | | | 2-8° C. | |
|---|---|---|---|---|---|---|
| Tests | Initial | 1 M | 3 M | 6 M | 3 M | 6 M |
| pH | 4.98 | 4.92 | 4.99 | 4.96 | 4.95 | 4.98 |
| Assay (%) | 102.7 | 103.2 | 103.4 | 105.1 | 103.3 | 105.8 |
| Related substances (%) | | | | | | |
| Impurity-C | ND | ND | ND | BQL (0.037) | ND | ND |
| Single max unknown Imp. | BQL (0.074) | 0.173 | 0.180 | 0.569 | 0.118 | 0.101 |
| Total Impurities | 0.115 | 0.290 | 0.297 | 0.678 | 0.118 | 0.152 |
| Osmolality (mosmol/Kg) | 293 | NA | NA | 355 | NA | 323 |

BQL: Below quantification level;
ND: Not detected;
NA: Not analyzed

The stability data indicates that the pH of the formulation remains within the desired range, and the assay of cetrorelix in the formulation is not less than 95%. Further, the solution remained as aggregation-free, clear colourless solution for at least six months under the storage conditions of 2° -8° C. and 25° C./60% RH.

### Example-5:

### Stability results for Impurity-C behavior in Reference Cetrotide® and the Cetrorelix acetate Injection (0.25 mg/0.5 ml) RTU formulation.

| | | | | | 2-8° C. | |
|---|---|---|---|---|---|---|
| | | 25° C./60% RH | | | | Near to expiry |
| Product details | Initial | 2 M | 3 M | 6 M | 6 M | samples |
| Cetrorelix RTU Injection of | ND | ND | ND | BQL (0.037) | ND | NA |
| the present invention | | | | | | |
| Cetrotide ® (Cetrorelix | 0.264% | NA | NA | 0.724% | NA | 0.507% |
| lyophilized injection; 0.25 | | | | | | |
| mg; Reference product) | | | | | | |

BQL: Below quantification level;
ND: Not detected;
NA: Not analyzed

The lyophilized powder for injection after reconstitution of Cetrotide® product showed increased levels of impurity-C, whereas the ready-to-use Cetrorelix formulation controlled the impurity-C below quantification level. The impurity-C of Cetrorelix is below 0.50% under the storage conditions of 2° -8° C. and 25° C./60% RH for at least six months. Therefore, the stable ready-to-use (RTU) cetrorelix formulation of the present invention exhibited superior impurity profile than the lyophilized product(Cetrotide®).

We claim:

1. A stable formulation of cetrorelix comprising:
a) cetrorelix or its pharmaceutically acceptable salt;
b) a cyclodextrin;
c) glacial acetic acid; and
d) a surfactant; wherein the said formulation can be administered as a ready-to-use solution.

2. The stable formulation as claimed in claim 1, wherein the pH of the said formulation is from about 3.5 to 8.5.

3. The stable formulation as claimed in claim 1, wherein cetrorelix or its pharmaceutically acceptable salt is in the concentration of about 0.01% to 10% W/V.

4. The stable formulation as claimed in claim 1, wherein the cyclodextrin is selected from α-Cyclodextrin, β-Cyclodextrin, γ-Cyclodextrin, Hydroxypropyl beta Cyclodextrin, Sulfobutyl-Ether beta Cyclodextrin or the likes thereof.

5. The stable formulation as claimed in claim 1, wherein the surfactant is selected from Polysorbate 80, Polysorbate 60, Polysorbate 20 or the likes thereof.

6. The stable formulation as claimed in claim 1, wherein the said formulation further comprises a tonicity adjusting agent selected from mannitol, dextrose, lactose, sucrose or the likes thereof.

7. A stable formulation of cetrorelix comprising cetrorelix acetate, hydroxypropyl beta cyclodextrin, polysorbate 80, glacial acetic acid, mannitol, and water for injection.

8. The stable formulation as claimed in claim 7, wherein the impurity-C of Cetrorelix is below 0.50% under the storage conditions of 2° -8° C. and 25° C./60% RH for at least six months.

9. The stable formulation as claimed in claim 7, wherein the said formulation remains as aggregation-free clear colourless solution under the storage conditions of 2° -8° C. and 25° C./60% RH for at least six months.

10. A process for preparation of a stable formulation comprises the steps of:
a) Transferring the water for injection into stainless steel vessel, and sparging nitrogen gas into the water.
b) Adding cyclodextrin to the solution and stiffing the solution,
c) Adding tonicity adjusting agent, surfactant, and cetrorelix acetate to the above bulk solution with stiffing until it gets dissolved,
d) Adjusting the desired pH of the bulk solution with glacial acetic acid,
e) Filtering the bulk solution, and filling the sterile solution into a suitable container.
